(19) United States
(12) Patent Application Publication
(10) Pub. No.: US 2026/0069657 A1
(43) Pub. Date: Mar. 12, 2026

(54) Haptoglobulin for Use in Treating or Preventing Exaggerated Erectile Response or Erectile Dysfunction

(71) Applicant: CSL Behring AG, Bern (CH)

(72) Inventors: Fábio Henrique Da Silva, Sumare-SP (BR); Fernando Ferreira Costa, Campinas-SP (BR)

(73) Assignee: CSL Behring AG, Bern (CH)

(21) Appl. No.: 19/107,058

(22) PCT Filed: Sep. 1, 2023
(86) PCT No.: PCT/EP2023/074009

§ 371 Date: Feb. 27, 2025

(30) Foreign Application Priority Data
Sep. 2, 2022 (EP) 22193626.3

(51) Int. Cl. A61K 38/17; A61K 48/00; A61P 15/00; A61P 15/10; C12N 15/86

(52) U.S. Cl. CPC A61K 38/1722; A61K 48/005; A61P 15/00; A61P 15/10; C12N 15/86; C12N 2750/14143

(57) ABSTRACT

The invention relates to haptoglobin or a nucleic acid encoding haptoglobin for use in treating or preventing of exaggerated erectile response and/or preventing permanent erectile dysfunction. In addition, the invention relates a pharmaceutical composition for use in treating or preventing exaggerated erectile response and/or preventing permanent erectile dysfunction, wherein the pharmaceutical composition comprises an adeno-associated viral (AAV) vector with a transgene encoding a haptoglobin gene. The exaggerated erectile response may be priapism, for example priapism associated with sickle cell disease (SCD).

### TECHNICAL FIELD

The invention relates to haptoglobin or a nucleic acid encoding haptoglobin for use in treating or preventing of exaggerated erectile response and/or preventing permanent erectile dysfunction. In addition, the invention relates to a pharmaceutical composition for use in treating or preventing exaggerated erectile response and/or preventing permanent erectile dysfunction, wherein the pharmaceutical composition comprises an adeno-associated viral (AAV) vector with a transgene encoding a haptoglobin gene. The exaggerated erectile response may be priapism, for example priapism associated with sickle cell disease (SCD).

### BACKGROUND OF THE INVENTION

Sickle cell disease (SCD), which is the most common single-gene disease, is caused by a point mutation in the β-globin gene. This alteration promotes the polymerization of hemoglobin under conditions of reduced oxygenation, causing rigid sickle-shaped red blood cells and hemolytic disease. SCD affects millions of people worldwide, approximately 100,000 Americans, and is most common in sub-Saharan Africa (1). Patients with SCD may exhibit exaggerated erectile response, for example priapism.

Priapism is characterized by prolonged, persistent and painful penile erection (2). The full or partial erection may continue hours beyond or isn't caused by sexual stimulation. An erection normally occurs in response to physical or psychological stimulation. This stimulation causes certain smooth muscles to relax, increasing blood flow to spongy tissues in the penis. As a result, the blood-filled penis becomes erect. After stimulation ends, the blood flows out and the penis returns to its nonrigid (flaccid) state. Priapism occurs when some part of this system—the blood, vessels, smooth muscles or nerves—changes normal blood flow, and an erection persists. The underlying cause of priapism often can't be determined, but several conditions may play a role. The main types of priapism are ischemic and nonischemic. Ischemic priapism is a medical emergency. Although priapism is an uncommon condition overall, it occurs commonly in certain groups, such as patients suffering from a blood disease such as sickle cell disease, leukemia or other blood diseases (hematologic dyscrasias), such as thalassemia and multiple myeloma. Prompt treatment for priapism is usually needed to prevent tissue damage that could result in the inability to get or maintain an erection (erectile dysfunction). Priapism most commonly affects males in their 30 s and older but can begin in childhood for males with sickle cell disease.

Recurrent events of priapism promote fibrosis in the erectile tissue that results in permanent erectile dysfunction (3). Erectile dysfunction (impotence) is the inability to get and keep an erection firm enough for sex. If erectile dysfunction is an ongoing issue, however, it can cause stress, affect one's self-confidence and contribute to relationship problems.

Clinical studies have shown that 30-45% of men with SCD have episodes of ischemic priapism and of these 30% progress to erectile dysfunction (4-6). Despite the high incidence of priapism, the main treatments available do not prevent priapism (2).

In SCD, the reduction in NO bioavailability is associated with intravascular hemolysis and increased oxidative stress (7). Increased production of reactive oxygen species in the penis is associated with increased gp91phox and p47phox subunits of NADPH oxidase, uncoupled eNOS and xanthine oxidase (8-10). Previous studies have shown that intravascular hemolysis may promote endothelial dysfunction associated with reduced eNOS activity in SCD mice (33). The reduced bioavailability of NO and increased oxidative stress in the penis has been identified as one of the main causes for triggering priapism in men and mice with SCD (8,11-13). Experimental evidences suggest that reduced NO/cGMP bioavailability results in a compensatory reduction in PDE5 activity and expression in the corpus cavernosum, thus impairing the mechanism that controls excessive penile erection (8,11,14).

Previous research studied the relationship between increased oxidative stress and the pathophysiology of priapism (8,12,29,40). Excess superoxide anion reacts with NO, forming peroxynitrite, which is a potent oxidizing agent (41). In addition, men and mice with SCD show lower expression and activity of the RhoA—Rho-kinase (ROCK) pathway, which participates in the contraction of corpus cavernosum smooth muscle and maintenance of the flaccid state of the penis (12,15). Some previous studies looked at the relationship between priapism and levels of intravascular hemolysis in men with SCD (20-22). Haptoglobin administration to SCD mice as well as its effects on blood vessels were also previously studied (23-25,39,46).

So far no definitive treatment strategy has been established to specifically treat patients with exaggerated erectile response or permanent erectile dysfunction, particularly priapism associated with sickle cell disease. It would therefore be highly desirable to provide an innovative approach for treating or preventing exaggerated erectile response and/or preventing permanent erectile dysfunction, in particular those associated with sickle cell disease.

### SUMMARY OF INVENTION

The present invention relates to haptoglobin, or a nucleic acid encoding it, for use in treating or preventing exaggerated erectile response and/or preventing permanent erectile dysfunction, preferably in human.

The haptoglobin may be a naturally occurring haptoglobin, for example a human haptoglobin, or a recombinant haptoglobin.

An exaggerated erectile response in the present context may be a prolonged erectile response. Preferably, the exaggerated erectile response is priapism, for example priapism associated with (caused by) sickle cell disease (SCD). Optionally, the haptoglobin increases PDE5 protein expression. Alternatively or additionally, the haptoglobin decreases exaggerated corpus cavernosum relaxations.

The permanent erectile dysfunction may be an aggravation of priapism associated with sickle cell disease (SCD). Preferably, the permanent erectile dysfunction occurs in patients suffering from SCD.

Preferably, the haptoglobin is a human plasma haptoglobin. Optionally, the haptoglobin or the nucleic acid encoding haptoglobin is administered parenterally, preferably by injection or infusion. Dosage of the haptoglobin or the nucleic acid encoding haptoglobin may be from 0.5 to 20 g per subject. It may also be administered based on the weight of the subject. Preferably, the haptoglobin or the nucleic acid encoding haptoglobin is administered three times a week, for a period of one month or longer.

According to another aspect of the present invention, it provides a pharmaceutical composition for use in treating or preventing exaggerated erectile response and/or preventing permanent erectile dysfunction. The exaggerated erectile response is preferably priapism, for example priapism associated with or caused by sickle cell disease (SCD). The permanent erectile dysfunction may be an aggravation of priapism associated with or caused by sickle cell disease (SCD). Preferably, the permanent erectile dysfunction occurs in patients suffering from SCD. The pharmaceutical composition comprises an adeno-associated viral (AAV) vector with a transgene encoding a haptoglobin gene. Optionally, the adeno-associated viral (AAV) vector comprises an AAV2 serotype, an AAV5 serotype, an AAV9 serotype, or a combination thereof.

The invention also relates to a method for treating or preventing exaggerated erectile response and/or preventing permanent erectile dysfunction. The method comprises administering a therapeutically effective amount of haptoglobin, or a nucleic acid encoding haptoglobin, or a pharmaceutical composition as described above, to a subject. Preferably, the subject is human.

### FIGURES

Specific embodiments of the present invention will be described by way of example, with reference to the accompanying drawings in which:

FIG. 1. Concentration-response curves to acetylcholine (ACh; A-B) and sodium nitroprusside (SNP, C-D) in corpus cavernosum strips from WT and SCD mice treated or not with haptoglobin (400 mg/kg) or vehicle every of Monday, Wednesday, and Friday for a period of 1 month. Data were calculated relative to the maximal changes from the contraction produced by phenylephrine (10⁻⁵ M) in each tissue, which was taken as 100%. Data represent the mean±SEM for 6 mice in each group. *P<0.05 vs WT-Vehicle.

FIG. 2. Relaxation responses induced by electrical-field stimulation (EFS) in corpus cavernosum strips WT and SCD mice treated or not with haptoglobin (400 mg/kg) or vehicle every of Monday, Wednesday, and Friday for a period of 1 month. Data were calculated relative to the maximal changes from the contraction produced by phenylephrine (10⁻⁵ M) in each tissue, which was taken as 100%. Data represent the mean±SEM for 6 mice in each group. *P<0.05 vs WT-Vehicle.

FIG. 3. Concentration-response curves to phenylephrine (A-B) and KCl (C-D) in corpus cavernosum strips from WT and SCD mice treated or not with haptoglobin (400 mg/kg) or vehicle every of Monday, Wednesday, and Friday for a period of 1 month. Data are shown in mN, and data represent the mean±SEM for 6 mice in each group. *P<0.05 vs WT-Vehicle.

FIG. 4. Representative images of western blotting (top panels) and protein values (bottom panels) for p-eNOS (Ser-1177; A), eNOS (B), nNOS (C) and PDE5 (D) in homogenates of penises from WT and SCD mice treated or not with haptoglobin (400 mg/kg) or vehicle every of Monday, Wednesday, and Friday for a period of 1 month. Data are shown in mN, and data represent the mean±SEM for 6 mice in each group. *P<0.05 vs WT-Vehicle. A single band for eNOS, nNOS and PDE5 was normalized to β-actin. A single band for p-eNOS (Ser-1177) was normalized to eNOS.

FIG. 5. Representative images of western blotting (top panels) and protein values (bottom panels) for ROCK2 (A) and ROCK1 (B) in homogenates of penises from WT and SCD mice treated or not with haptoglobin (400 mg/kg) or vehicle every of Monday, Wednesday, and Friday for a period of 1 month. Data are shown in mN, and data represent the mean±SEM for 6 mice in each group. *P<0.05 vs WT-Vehicle. A single band for ROCK1 and ROCK2 was normalized to β-actin.

FIG. 6. Representative images of western blotting (top panels) and protein values (bottom panels) for gp91phox (A), 3-NT (B) and 4-HNE (C) in homogenates of penises from WT and SCD mice treated or not with haptoglobin (400 mg/kg) or vehicle every of Monday, Wednesday, and Friday for a period of 1 month. Data are shown in mN, and data represent the mean±SEM for 6 mice in each group. *P<0.05 vs WT-Vehicle. A single band for gp91phox, 3-NT and 4-HNE was normalized to β-actin.

FIG. 7. Basal cGMP contents of penis from WT and SCD mice treated or not with haptoglobin. Data represent the mean±S.E.M. for 6 mice in each group. *P<0.05 vs WT-Vehicle; #P<0.05 vs SCD-Hap.

### DETAILED DESCRIPTION

The process of intravascular hemolysis leads to the release of hemoglobin into the plasma (16). Under physiological conditions, haptoglobin is the plasma protein responsible for defending the body against the accumulation of free hemoglobin (17). In plasma, haptoglobin binds to free hemoglobin forming a hemoglobin-haptoglobin complex that is metabolized by macrophages in the reticuloendothelial system. Macrophages express the CD163 receptor that mediates endocytosis and internalization of the hemoglobin-haptoglobin complex (17). However, in SCD, high concentrations of hemoglobin are released into the plasma, depleting haptoglobin, and thus accumulating free hemoglobin in the plasma (16,18,19). Hemoglobin in plasma or interstitial space reacts with NO, generating nitrate and methemoglobin (16). Elevated plasma hemoglobin and heme levels promote vascular and oxidative damage in SCD (37-39).

Without wishing to be bound by theory, the inventors of the present invention have found that, surprisingly, treatment with haptoglobin may reverse the exaggerated erectile response, in particular the exaggerated erectile response induced by stimulation of the NO-cGMP pathway, by normalizing eNOS and PDE5 expression, as well as normalizing the contractile activity of corpus cavernosum and increased oxidative stress.

Without wishing to be bound by theory, it is believed that the improvement was achieved by up-regulation of eNOS-PDE5 expression and down-regulation of the gp91phox subunit of NADPH oxidase and oxidative/nitrosative stress in the penis.

The haptoglobin may decrease exaggerated corpus cavernosum relaxations. Alternatively or additionally, the haptoglobin may increase PDE5 protein expression. Phosphodiesterase 5 (PDE5) is a multidomain protein that functions as a dimer to hydrolyze cGMP. PDE5 expression is positively regulated by basal levels of cGMP in the penis (30). Penises from men and mice with SCD display lower basal production of NO due to lower expression and activity of eNOS (9,10,12,31). The lower bioavailability of NO results in reduced activation of sGC, a heterodimeric enzyme that contains a heme group that catalyzes the synthesis of the second messenger cGMP (32). Low expression of PDE5 in the penis may contribute to the increased relaxation of corpus cavernosum (8,29). The inventors of the present invention have found that, surprisingly, haptoglobin treatment may help increase PDE5 protein expression, thereby treating or preventing exaggerated erectile response.

### Haptoglobin and Variants Thereof

Haptoglobin (Hp) is a tetrameric plasma glycoprotein of a molecular weight of approximately 90,000 kDa. Produced by hepatocytes, its main function is to prevent iron loss due to hemolysis of erythrocyte, through its affinity and binding capacity to free hemoglobin (Hb). Hp is synthesised as a single chain, which is post-translationally cleaved into an amino-terminal α chain and a carboxy-terminal β chain. The basic structure of Hp, as found in most mammals, is a homodimer, in which the two Hp molecules are linked by a single disulfide bond via their respective 9 kDa α chains. In man, a variant with a long α chain is also present in all populations. This variant arose apparently by an early intragenic duplication, presumably originating from an unequal crossover of two basic alleles, resulting in an Hp with an α chain of 14 kDa. The short and long α chains are designated as α1 and α2 respectively. Since the cysteine forming the intermolecular disulfide bond between the α chains is also duplicated, humans carrying the long variant allele exhibit a multimeric Hp phenotype. There are two isoforms of the α chain determining the existence of subtypes within Hp phenotypes Hp 2-1 and Hp 1-1. Isoforms α-1f and α-1s differ in amino acid sequence only at the positions 52 and 53, where the chain α-1f contains aspartic acid and lysine while chain α-1s presents two residues, one asparagine and one glutamic acid (47). These different isoforms share essentially equivalent characteristics of hemoglobin affinity, binding and clearance, with only small nuanced differences in function.

In vivo, haptoglobin is synthesized as a single polypeptide precursor exhibiting a molecular weight of 38,000 kDa. It is thought that all three phenotypes of the mature protein are derived from a single precursor, haptoglobin-1 precursor. The polypeptide precursor is proteolytically processed to form the α and β-subunits of the native protein (48). The precursor protein includes an amino-terminal 18 residue signal sequence before the α chain, and/or an intervening polypeptide between the α and β-regions (49). In vivo, post-translational events result in the proteolytic removal of the signal sequence and the incorporation of the core oligosaccharide side chains into the β-region by membrane-associated enzyme systems (48). Post-translational modification may also result in the cleavage of both α and β-regions of the precursor polypeptide to form the native protein (48).

It should be understood that naturally occurring and recombinant forms of Hp are suitable for the use of the present invention, as long as they can form a complex with cell-free Hb to neutralize the biological activity of cell-free Hb. Suitable naturally occurring forms of Hp are known to those skilled in the art, illustrative examples of which include Koch et al. (50) and Kasvosve et al. (51), the entire contents of which are incorporated herein by reference.

Optionally, the haptoglobin comprises, consists of, or consists essentially of plasma derived Hp. Preferably, the haptoglobin is a human plasma haptoglobin. Preferably, the haptoglobin is a human haptoglobin, such as protein disclosed under NCBI accession number NP_005134 or UniProt accession number P00738. Various variants are available at that UniProt entry. Protocols for isolating Hp from natural sources of Hp (e.g., plasma) will be familiar to those skilled in the art.

The sequence of the β chain of human haptoglobin may be:

(SEQ ID NO: 3)
ILGGHLDAKGSFPWQAKMVSHHNLTTGATLINEQWLLTTAKNLFL
NHSENATAKDIAPTLTLYVGKKQLVEIEKWVLHPNYSQVDIGLIK
LKQKVSVNERVMPICLPSKDYAEVGRVGYVSGWGRNANFKFTDHL
KYVMLPVADQDQCIRHYEGSTVPEKKTPKSPVGVQPILNEHTFCA
GMSKYQEDTCYGDAGSAFAVHDLEEDTWYATGILSFDKSCAVAEY
GVYVKVTSIQDWVQKTIAEN

The sequence of the α2 chain of human haptoglobin may be:

(SEQ ID NO: 4)
VDSGNDVTDIADDGCPKPPEIAHGYVEHSVRYQCKNYYKLRTEGD
GVYTLNDKKQWINKAVGDKLPECEADDGCPKPPEIAHGYVEHSVR
YQCKNYYKLRTEGDGVYTLNNEKQWINKAVGDKLPECEAVCGKPK
NPANPVQ

The sequence of the α1 chain of human haptoglobin may be:

(SEQ ID NO: 5)
VDSGNDVTDIADDGCPKPPEIAHGYVEHSVRYQCKNYYKLRTEGD
GVYTLNNEKQWINKAVGDKLPECEAVCGKPKNPANPVQ

The canonical sequence of α2-β of human haptoglobin is shown as Isoform 1 in P00738 (last update: 1986-07-21). The sequence of Isoform 1 is:

(SEQ ID NO: 1)
MSALGAVIALLLWGQLFAVDSGNDVTDIADDGCPKPPEIAHGYVE
HSVRYQCKNYYKLRTEGDGVYTLNDKKQWINKAVGDKLPECEADD
GCPKPPEIAHGYVEHSVRYQCKNYYKLRTEGDGVYTLNNEKQWIN
KAVGDKLPECEAVCGKPKNPANPVQRILGGHLDAKGSFPWQAKMV
SHHNLTTGATLINEQWLLTTAKNLFLNHSENATAKDIAPTLTLYV
GKKQLVEIEKVVLHPNYSQVDIGLIKLKQKVSVNERVMPICLPSK
DYAEVGRVGYVSGWGRNANFKFTDHLKYVMLPVADQDQCIRHYEG
STVPEKKTPKSPVGVQPILNEHTFCAGMSKYQEDTCYGDAGSAFA
VHDLEEDTWYATGILSFDKSCAVAEYGVYVKVTSIQDWVQKTIAE
N

The sequence of an isoform of human haptoglobin (α1-β) which occurs due to alternative splicing is shown as Isoform 2 in P00738. The sequence differs from canonical Isoform 1 in that amino acids 38-96 are missing. The sequence of Isoform 2 may be:

(SEQ ID NO: 2)
MSALGAVIALLLWGQLFAVDSGNDVTDIADDGCPKPPEIAHGYVE
HSVRYQCKNYYKLRTEGDGVYTLNNEKQWINKAVGDKLPECEAVC
GKPKNPANPVQRILGGHLDAKGSFPWQAKMVSHHNLTTGATLINE
QWLLTTAKNLFLNHSENATAKDIAPTLTLYVGKKQLVEIEKVVLH
PNYSQVDIGLIKLKQKVSVNERVMPICLPSKDYAEVGRVGYVSGW
GRNANFKFTDHLKYVMLPVADQDQCIRHYEGSTVPEKKTPKSPVG
VQPILNEHTFCAGMSKYQEDTCYGDAGSAFAVHDLEEDTWYATGI
LSFDKSCAVAEYGVYVKVTSIQDWVQKTIAEN

As used herein, the term “haptoglobin” is to be understood to encompass all phenotypes (including all isoforms) of Hp. The haptoglobin may be homologous (so long as it consists essentially of Hp of the same isoform) or heterologous (including combinations of different Hp isoforms, including Hp1-1, Hp1-2 and Hp2-2). Optionally, the haptoglobin for use according to the present invention may be a mixture of two or more phenotypes and/or isoforms. Preferably, the haptoglobin for use according to the present invention comprises Hp2-2. More preferably, the haptoglobin for use according to the present invention is a mixture of Hp2-2 and Hp 1-2. It should be understood that the composition of Hp ultimately depends on the phenotype of the source. For example, if a pooled plasma sample is used to extract/purify Hp, it is likely that one or more isoforms of Hp will be isolated. Suitable methods for determining the Hp isoforms present in an isolate will be familiar to those skilled in the art.

In one embodiment, Hp is selected from the group consisting of Hp1-1 homodimer, Hp1-2 multimer, Hp2-2 multimer and combinations thereof. The Hp may be naturally occurring Hp (e.g., derived from plasma) or it may be produced as a recombinant protein. In some embodiments, the haptoglobin comprises naturally occurring human plasma haptoglobin. Optionally, the plasma-derived Hp comprises, consists of, or consists essentially of Hp2-2. Alternatively, the plasma-derived Hp comprises, consists of, or consists essentially of Hp1-1. In a further aspect, the Hp comprises, consists of, or consists essentially of a recombinant Hp.

The term Hp as used herein includes functional analogues of naturally occurring or naturally occurring Hp. The term “functional analogue” is intended to mean an agent that shares substantially identical biological activity as a naturally occurring (native) Hp, so long as the biological activity is at least the ability of the analogue to form a complex with a cell-free Hb to neutralize its biological activity.

The term “substantially identical biological activity” typically means that the functional analogue has at least 40% of the binding affinity to Hb (for example, 40%, 45%, 50%, 55%, 60%, 65%, 70%, 85%, 90%, 95%, 100%, 105%, 110%, 115%, 120%, 125%, 130%, 135%, 140%, 145%, 150%, 155%, 160%, 165%, etc.). Suitable methods for determining whether an agent is a functional analog of Hp will be familiar to those skilled in the art.

Optionally, the functional analog of Hp is a functional fragment of native Hp. A functional fragment of native Hp can be of any suitable length so long as the fragment retains the ability to form complexes with cell-free Hb and neutralize its biological activity.

Alternatively, the functional analog is a polypeptide having an amino acid sequence that differs from the naturally occurring (native) Hp molecule (i.e., comparator). A functional analogue differs from the amino acid sequence of the alpha and/or beta chain of a native Hp by one or more (e.g., 1, 2, 3, 4, 5, 6, 7, 8, 9 or more) amino acid substitutions having different amino acid sequences, wherein the difference does not abrogate or completely abolish the ability of the analog to form complexes with cell-free Hb and neutralize its biological activity. In some embodiments, the functional analog comprises an amino acid substitution that enhances the ability of the analog to form a complex with a cell-free Hb, as compared to native Hp. In one embodiment, the functional analog has an amino acid sequence that differs from the amino acid sequence of the alpha and/or beta chain of a native Hp by one or more conservative amino acid substitutions. As used herein, the term “conservative amino acid substitution” refers to altering the amino acid identity at a given position and replacing it with an amino acid of approximately equivalent size, charge and/or polarity. Examples of naturally conservative substitutions of amino acids include the following groups of eight substituents (designated by the general one-letter code): (1) M, I, L, V; (2) F, Y, W; (3) K, R, (4) A, G; (5) S, T; (6) Q, N; (7) E, D; and (8) C, S.

In one embodiment, the functional analogue has at least 85% or greater sequence identity with the amino acid sequence of the alpha and/or beta chain of native Hp.

Reference to “at least 85%” means, for example, after best alignment or best fit analysis, 85%, 86%, 87%, 88%, 89%, 90%, 91%, 92%, 93%, 94%, 95%, 96%, 97%, 98%, 99% or 100% sequence identity or similarity. Thus, in one embodiment, the sequence has at least 85%, at least 86%, at least 87%, at least 87%, at least 85%, at least 86%, at least 88%, at least 89%, at least 90%, at least 91%, at least 92%, at least 93%, at least 94%, at least 95% %, at least 96%, at least 97%, at least 98%, at least 99% or 100% sequence identity or sequence homology with the amino acid sequence of the alpha and/or beta chain of native Hp.

In the present text, the term “percentage identity” and “% identity” between two amino acid (peptide) or nucleic acid (nucleotide) sequences means the percentage of identical amino acid or nucleotide residues in corresponding positions in the two optimally aligned sequences. No conservative substitutions are considered as part of identity. Neither N- or C-terminal extensions or insertions should be construed as reducing sequence identity or homology.

To determine the “percentage identity” of the two amino acid or nucleic acid sequences, the sequences are aligned together. To achieve an optimal match, gaps can be introduced into the sequence (i.e. deletions or insertions which can also be placed at the sequence ends). Amino acid and nucleotide residues in the corresponding positions are then compared. When a position in the first sequence is occupied by the same amino acid or nucleotide residue that occupies the corresponding position in the second sequence, the molecules are identical in that position. The percentage identity between two sequences is a function of the number of identical positions divided by the sequences [i.e. % identity=(number of identical positions/total number of positions)×100].

In the present text, the term “similarity” or “sequence similarity” indicates that, at any particular position in an aligned sequence, amino acid residues are of a similar type between sequences. For example, leucine may be substituted with an isoleucine or valine residue. As noted elsewhere herein, this may be referred to as a conservative substitution. In one embodiment, the amino acid sequence is modified by conservative substitution of any amino acid residues contained therein such that the modification does not affect the binding specificity or functional activity of the modified polypeptide as compared to the unmodified (native) Hp polypeptide.

The percentage identity can be obtained by using mathematical algorithms. A non-limiting example of an algorithm used for comparing two sequences is incorporated in the BLASTn and BLASTp programs of Altschul [52]. Those programs can also be used to achieve alignments even in the presence of one or more gaps (for insertions). For this purpose the BLASTn and BLASTp programs can be used with the default parameters (i.e. for amino acid sequence a penalty of −12 for a gap and a penalty of −4 for each extension are allowed). When using the BLAST program the BLOSUM62 matrix is typically employed.

In one embodiment, a functional analog comprises amino acid substitutions and/or other modifications relative to native Hp to increase the stability of the analog or increase the solubility of the analog.

Functional analogs may be naturally occurring polypeptides or may be produced synthetically by chemical synthesis using methods known to those skilled in the art.

Hp may be suitably produced as a recombinant protein in a microorganism, which may be isolated and, if desired, further purified. Microorganisms suitable for the production of recombinant Hp will be familiar to those skilled in the art, illustrative examples of which are bacteria, yeast or fungi, eukaryotic cells (eg mammalian or insect cells), or recombinant viral vectors (e.g. adenovirus, poxvirus, herpesvirus, shimki forest fever virus, baculovirus, bacteriophage, Sindbis virus or Sendai virus). Bacteria suitable for producing recombinant peptides will be familiar to those skilled in the art, illustrative examples of which include E. coli, B. subtilis, or any other bacteria capable of expressing a peptide sequence. Illustrative examples of suitable yeast types for producing recombinant peptides include Candida (Candida), blood Chiapas Pastoris (Pichia pastoris), Saccharomyces cerevisiae, Schizosaccharomyces pombe, or any other yeast capable of expressing peptides. Such methods are well known in the art. Methods for isolating and purifying recombinantly produced peptide sequences are also well known in the art and include, for example, gel filtration, affinity chromatography, and ion exchange chromatography.

To facilitate isolation of recombinant Hp as described herein, a fusion polypeptide in which a peptide sequence of Hp or a functional analog thereof is translationally fused (covalently bound) to a heterologous polypeptide allowing isolation by affinity chromatography can be manufactured. Illustrative examples of suitable heterologous polypeptide are a His-tag (for example, His 6: 6 histidine residues), a GST-tag (glutathione transferase -S-) etc.

For the production of recombinant Hp, phage libraries and/or peptide libraries are also suitable, e.g., generated by binding chemistry or obtained by high-throughput screening techniques for the most diverse structures.

Illustrative examples of recombinant Hp are NCBI accession number NP_005134 (as described by Morishita et al (53)) and UniProt accession number P00738.

### Half-Life Extending Haptoglobin Variants

The haptoglobin according to the present invention may be fused, coupled or attached to one or more heterologous moieties as part of a fusion protein. One or more heterologous moieties may improve, enhance or extend the activity or stability of Hp. In one embodiment, the Hp is suitably attached to a heterologous moiety to extend the half-life of the Hp in vivo. Suitable half-life extending heterologous moieties will be familiar to those skilled in the art, illustrative examples of which are polyethylene glycol (PEGylation), glycosylated PEG, hydroxyl ethyl starch (HESylation), polysialic acid, elastin-like polypeptide, heparo acid polymers and hyaluronic acid. Thus, in some embodiments disclosed herein, the heterologous moiety is selected from the group consisting of polyethylene glycol (PEGylated), glycosylated PEG, hydroxyl ethyl starch (HESylation), polysialic acid, elastin-like polypeptide, heparoic acid polymer, and hyaluronic acid. In another embodiment, the heterologous moiety may be a heterologous amino acid sequence fused to Hp.

Alternatively, or in addition, the heterologous moiety may be chemically conjugated, e.g. covalently linked, to the Hp. The half-life extending heterologous moiety may be fused, conjugated or otherwise attached to the Hp by any suitable means known to those skilled in the art, illustrative examples of which are via a chemical linker. The principles of this bonding technique have been described by way of example by Conjuchem LLC (see, e.g. U.S. Pat. No. 7,256,253), the entire contents of which are incorporated herein by reference.

In another embodiment, the heterologous moiety is a half-life enhancing protein (HLEP). Suitable half-life enhancing proteins will be familiar to those skilled in the art, illustrative examples of which include albumin or fragments thereof. Thus, in one embodiment, the HLEP is albumin or a fragment thereof. The N-terminus of the albumin or fragment thereof may be fused to the C-terminus of the alpha and/or beta chain of Hp. Alternatively or additionally, the C-terminus of the albumin or fragment thereof may be fused to the N-terminus of the alpha and/or beta chain of Hp. One or more HLEPs may be fused to the N- or C-terminal portion(s) of the alpha and/or beta chain of the Hp so long as it does not abrogate the binding of the Hp to the cell-free Hb. However, it should be understood that a slight decrease in binding of Hp to cell-free Hb may be acceptable as long as the Hp component of the fusion protein can still form a complex with cell-free Hb to neutralize cell-free Hb.

The fusion protein may further comprise a chemical bond or linker sequence positioned between the Hp and the heterologous moiety. The linker sequence comprises at least one amino acid, in particular 1 to 50, preferably 1 to 30, preferably 1 to 20, preferably 1 to 15, preferably 1 to 10, preferably 1 to 5. It may be a peptide linker of 5 or more preferably 1 to 3 (e.g. 1, 2 or 3) amino acids, which may be identical to or different from each other. Preferably, the linker sequence is not present in the corresponding position of wild-type Hp. Preferred amino acids present in the linker sequence include Gly and Ser. In a preferred embodiment, the linker sequence is substantially non-immunogenic to the subject to be treated according to the methods disclosed herein. Substantially non-immunogenic means that the linker sequence will not elicit a detectable antibody response to the linker sequence in the subject to which it is administered. Preferred linkers may consist of alternating glycine and serine residues. Suitable linkers will be familiar to those skilled in the art, illustrative examples of which are described in WO 2007/090584. In one embodiment, the peptide linker between the Hp and the heterologous moiety comprises, consists of, or consists essentially of a peptide sequence that acts as a native interdomain linker in a human protein. In the natural environment, these peptide sequences can be located close to the protein surface and have access to the immune system, so a natural resistance to these sequences can be assumed. Illustrative examples are provided in WO 2007/090584. Suitable cleavable linker sequences are described, for example, in WO 2013/120939 A1.

Illustrative examples of suitable HLEP sequences are described below. Likewise fusions to the exact “N-terminal amino acid” or to the exact “C-terminal amino acid” of the respective HLEP, or fusions to the “N-terminal part” or “C-terminal part” of the respective HLEP, which includes N-terminal deletions of one or more amino acids of the HELP, are also described. The fusion protein may comprise one or more HLEP sequences, e.g. two or three HLEP sequences. Such multiple HLEP sequences may be fused to the C-terminal portion of the alpha and/or beta chain of Hp in tandem, e.g. in successive repeats.

In one embodiment, the heterologous moiety is a half-life extending polypeptide. In one embodiment, the half-life extending polypeptide comprises albumin, a member of the albumin family or fragment thereof, a solvated random chain with large hydrodynamic volume (e.g. XTEN (56), homo-amino acid repeat (HAP) or proline-alanine-serine repeat (PAS), apamine, alpha-fetoprotein, vitamin D binding protein, transferrin or variants or fragments thereof, human chorionic gonadotropin carboxyl-terminal peptide (CTP) of the β subunit, neonatal Fc receptor (FcRn), in particular immunoglobulin constant region and parts thereof, e.g., polypeptides capable of binding to Fc fragments. It is selected from the group consisting of a member of the family or a fragment thereof or a polypeptide or lipid capable of binding to an immunoglobulin constant region or a part thereof. The immunoglobulin constant region or a part thereof is preferably an Fc fragment of immunoglobulin G1 (IgG1), an Fc fragment of immunoglobulin G2 (IgG2), or an Fc fragment or immunoglobulin a (IgA). A HLEP may be a full-length half-life-enhancing protein or one or more fragments thereof that are capable of stabilizing or prolonging the therapeutic activity or the biological activity of the Hp, in particular of increasing the in vivo half-life of the Hp. This fragment can be more than 10 amino acids in length, or at least about 15, preferably at least about 20, preferably at least about 25, preferably at least about 30, preferably at least about 50, or more preferably at least about 100, or more contiguous amino acids from the HLEP sequence, or may include part or all of specific domains of the respective HLEP, as long as the HLEP fragment provides a functional half-life extension of at least 10%, preferably of at least 20%, or more preferably of at least 25%, compared to the Hp in the absence of the HLEP. Methods for determining whether a heterologous moiety provides a functional half-life extension to Hp (in vivo or in vitro) will be familiar to those skilled in the art.

The HLEP portion of the fusion protein as described herein may be a variant of wild-type HLEP. The term “variant” includes insertions, deletions and/or substitutions, whether conservative or non-conservative, wherein such changes do not substantially alter the ability of the Hp to form a complex with the cell-free Hb to neutralize the cell-free Hb. HLEP may suitably be derived from any vertebrate, in particular any mammal, such as a human, monkey, cattle, sheep or pig. Non-mammalian HLEPs include, but are not limited to, hens and salmon.

Fusion proteins as described herein can be produced by in-frame joining of at least two DNA sequences encoding heterologous moieties such as Hp and HLEP. Those skilled in the art will understand that translation of the fusion protein DNA sequence will result in a single protein sequence. As a result of in-frame insertion of a DNA sequence encoding a peptide linker according to aspects disclosed herein, a fusion protein comprising Hp, a suitable linker and a heterologous moiety can be obtained.

As used herein, “albumin” refers collectively to an albumin polypeptide or amino acid sequence, or albumin fragment or variant, having one or more functional (e.g., biological) activities of albumin. In particular, “albumin” refers to human albumin or fragments thereof, including mature forms of human albumin, or albumin or fragments thereof from other vertebrates, or analogs or variants of these molecules or fragments thereof. In some aspects disclosed herein, the alternative term “FP” is used to identify HLEP, and in particular is used to define albumin as HLEP.

The fusion proteins described herein may suitably comprise naturally occurring polymorphic variants of human albumin and/or fragments of human albumin. Generally speaking, an albumin fragment or variant will be at least 10 amino acids in length, preferably at least 40, or most preferably at least 70 amino acids.

In one embodiment, the HLEP is an albumin variant with enhanced binding to the FcRn receptor. Such albumin variants may result in a longer plasma half-life of Hp or a functional analog thereof compared to Hp or a functional fragment thereof fused to wild-type albumin.

The albumin portion of the fusion proteins described herein may suitably comprise at least one subdomain or domain of human albumin or conservative modifications thereof.

In one embodiment, the heterologous moiety is an immunoglobulin molecule or functional fragment thereof. Immunoglobulin G (IgG) constant regions (Fc) are known in the art to increase the half-life of therapeutic proteins (57). The IgG constant region of the heavy chain consists of three domains (CH1-CH3) and a hinge region. The immunoglobulin sequence may be derived from any mammal, or from subclasses IgG1, IgG2, IgG3 or IgG4, respectively. IgG and IgG fragments lacking antigen-binding domains can also be used as heterologous moieties, including as HLEPs. The Hp or functional analog thereof may be suitably linked to the IgG or IgG fragment via the hinge region of the antibody or via a peptide linker which may be cleavable. Several publications describe the fusion of a therapeutic protein to an immunoglobulin constant region to enhance the in vivo half-life of the therapeutic protein. For example, US 2004/0087778 and WO 2005/001025 disclose fusion proteins of a biologically active peptide that increases the half-life of the peptide and is otherwise rapidly cleared in vivo with at least a portion of an Fc domain or immunoglobulin constant region. Fc-IFN-β fusion proteins that achieve enhanced biological activity, extended circulating half-life and greater solubility have been described (WO 2006/000448 A2). Fc-EPO proteins with a prolonged serum half-life and increased in vivo potency were disclosed (WO 2005/063808 A1) as well as Fc fusions with G-CSF (WO 2003/076567 A2), glucagon-like peptide-1 (WO 2005/000892 A2), clotting factors (WO 2004/101740 A2) and interleukin-10 (U.S. Pat. No. 6,403,077), all with half-life enhancing properties.

Illustrative examples of suitable HLEPs that may be used in accordance with the present invention are also described in WO 2013/120939 A1, the content of which is incorporated herein by reference in its entirety.

### Nucleic Acids Encoding Haptoglobin

As an alternative to haptoglobin, a nucleic acid encoding haptoglobin may be used in the present invention. The haptoglobin may be any of the haptoglobins described above, including variants, fragments and fusion proteins thereof.

Haptoglobin in humans is encoded by the HP gene, for example NCBI Accession No. NP_001119574. NP_001305067, NP_005134. Two major alleles, Hp1 and Hp2, exist for the Hp gene found on chromosome 16. The two alleles are responsible for three different possible genotypes with structural polymorphisms: homozygous (1-1 or 2-2) and heterozygous 2-1.

The term “nucleic acid” as used herein generally relates to any nucleotide molecule which encodes the haptoglobin according to the invention and which may be of variable length. Examples of a nucleic acid of the invention include, but are not limited to, plasmids, vectors, or any kind of DNA and/or RNA fragment(s). Nucleic acid molecules for use of the present invention may be in the form of RNA, such as mRNA or cRNA, or in the form of DNA, including, for instance, cDNA and genomic DNA, e.g. obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand. Nucleic acid molecule as used herein also refers to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA. Furthermore, the nucleic acid molecule encoding the haptoglobin according to the invention can be functionally linked, using standard techniques such as standard cloning techniques, to any desired sequence, such as a regulatory sequence, leader sequence, heterologous marker sequence or a heterologous coding sequence to create a fusion protein.

The nucleic acid according to the present invention may be present together with appropriate promotor-, enhancer-, marker-, etc. sequences e.g. in a vector, such as a plasmid or viral vector, allowing expression of the polypeptide or the mRNA in a target cell, tissue or body fluid. The present nucleic acids may also encompass nucleic acid sequences which encode haptoglobin receptors or haptoglobin binding fragments thereof.

### Gene Therapy

According to another aspect of the invention, it relates to a pharmaceutical composition for use in treating or preventing exaggerated erectile response and/or preventing permanent erectile dysfunction comprising an adeno-associated viral (AAV) vector with a transgene encoding a haptoglobin gene. In the context of the present invention, a haptoglobin gene is any gene that encodes a haptoglobin as described herein.

Numerous (viral) delivery systems have been investigated, all of them with their advantages and drawbacks. One of the viral delivery vehicles that are used for gene therapy is the Adeno Associated Virus (AAV).

AAV has a single-stranded DNA genome of approximately 4.8 kilobases (kb). AAV belongs to the parvovirus family and is dependent for replication on co-infection with other viruses, in particular adenoviruses. The genome comprises Rep (Replication) and Cap (Capsid) genes. These coding sequences are flanked by inverted terminal repeats (ITRs) that are required for genome replication and packaging. The Rep gene encodes four proteins (Rep78, Rep68, Rep52, and Rep40), replicates the viral genome, and facilitates packaging, while Cap expression gives rise to the viral capsid proteins (VP; VP1 VP2 VP3), which form the outer capsid shell.

Recombinant AAV (rAAV) for gene therapy is formed by a protein capsid containing a desired nucleic acid, the transgene, that is to be delivered to target cells. The desired nucleic acid is flanked by the ITRs of AAV. ITR-flanked transgenes encoded by rAAV can form circular concatemers remaining in the nucleus of transduced cells as episomes. As the episome remains largely episomal, the expression of AAV delivered nucleic acid sequences may be diluted over time if and when the target cell replicates. This dilution may not generally apply to post-mitotic cells such as neurons, which are the target cells for many neurodegenerative diseases. A review on AAV vectors for gene therapy is provided in Naso et al., Biodrugs 2017 (p.317-334).

In some embodiments, the adeno-associated viral vector comprises an AAV2 serotype, an AAV5 serotype, an AAV9 serotype, a hybrid AAV serotype, or a combination thereof. In some embodiments, the adeno-associated viral vector comprises an AAV5 serotype. In some embodiments, the adeno-associated viral vector comprises an AAV9 serotype.

In some embodiments, the adeno-associated viral vector comprises a hybrid AAV serotype. By way of example, the hybrid AAV serotype may be a hybrid AAV2/AAV5; AAV2/AAV9; or AAV5/AAV9 serotype.

### Administration

The haptoglobin or the nucleic acid of the present invention be administered to the subject in a dosage form of, for example, a lyophilized formulation or an aqueous solution. The dosage form may further comprise one or more optional pharmaceutically acceptable carriers. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). The haptoglobin or the nucleic acid of the present invention as active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. The dosage forms to be used for in vivo administration are generally sterile. Sterility may be readily accomplished, e.g. by filtration through sterile filtration membranes.

Preferably, the haptoglobin or the nucleic acid encoding haptoglobin of the present invention is administered parenterally, more preferably by injection or infusion. For example, the haptoglobin or the nucleic acid encoding haptoglobin is administered systemically, for example by intravenous administration, such as intravenous injection or infusion. Alternatively, the haptoglobin or the nucleic acid encoding haptoglobin is administered by subcutaneous injection. The dosage of Hp may also be adjusted to provide an optimal therapeutic response. For example, several divided doses may be administered daily, weekly, or other suitable time intervals, or the dosage may be proportionally reduced according to the urgency of the situation. Optionally, the haptoglobin or the nucleic acid encoding haptoglobin is administered three times a week, for a period of one month or longer. Alternatively, the haptoglobin or the nucleic acid encoding haptoglobin may be administered for a period of one to three months. Preferably, the haptoglobin or the nucleic acid encoding haptoglobin is administered according to a repeat pattern of two 48-h and one 72-h dosing intervals.

The administration routes described above may also apply to the pharmaceutical composition of the present invention.

As used herein, the term “therapeutically effective amount” refers to an amount or concentration of Hp sufficient to neutralize adverse biological effects of cell-free Hb by binding to and complexing with cell-free Hb present in plasma. It will be understood by those skilled in the art that a therapeutically effective amount of a peptide may vary depending on several factors, illustrative examples of which include the route of administration, the health and physical condition of the subject being treated, the taxonomic group of the subject being treated, the severity of the bleeding (e.g. the degree of bleeding), the concentration and/or amount of acellular Hb in the plasma, and any of the foregoing. Combinations of any are included.

A therapeutically effective amount of Hp will typically fall within a relatively wide range that can be determined by one of ordinary skill in the art. Optionally, the therapeutically effective amount of Hp is from about 0.5 g to about 50 g, from about 0.5 g to about 40 g, from about 0.5 g to about 30 g, from about 0.5 g to about 20 g, from about 0.5 g to about 15 g, from about 0.5 g to about 10 g, per subject. Preferably, the haptoglobin or the nucleic acid encoding haptoglobin may be administered at 1 g to 5 g, more preferably from 1.5 g to 3 g, per subject. The Hp to be administered may be in a concentration of from about 2 μM to about 20 mM, from about 2 μM to about 5 mM, from about 2 μM to about 300 μM, from about 5 μM to about 100 μM, about 5 μM to about 50 μM, or about 5 μM to about 30 μM, and the administrated amount may be from 1 to 50 mL, from 1 to 40 mL, from 1 to 30 mL, from 3 to 20 mL, for example from 5 to 10 mL, per subject. Alternatively, a therapeutically effective amount of Hp may be based on the body weight of the subject. For example, the therapeutically effective amount of Hp may be from 1 to 100 mg/kg weight of the subject, optionally from 1 to 80 mg/kg, from 5 to 75 mg/kg, from 10 to 50 mg/kg, from 15 to 50 mg/kg, from 20 to 40 mg/kg, or from 25 to 35 mg/kg weight of the subject.

In some embodiments, the haptoglobin or the nucleic acid encoding haptoglobin is administered in a dosage of from 5 to 10 mL at a concentration of from 50 to 220 mg/dL (from 5.88 to 25.87 μM) per subject. In another embodiment, the therapeutically effective amount of Hp is an amount sufficient to form a complex with about 3 μM to about 300 μM, about 5 μM to about 250 μM, about 10 μM to about 200 μM, about 50 μM to about 150 μM, cell-free Hb in plasma. Suitable methods for determining the concentration of cell-free Hb in CSF are known to those skilled in the art, illustrative examples of which are described by Cruickshank et al (54) and Hugelshofer M. et al (55), the contents of which are incorporated herein by reference in their entirety.

For the pharmaceutical composition of the present invention, it may be administered in a dosage of about 1E12 to about 5E14, about 5E12 to about 1E14, or about 1E13 to about 1E14 genome copies of the adeno-associated viral (AAV) vector per subject.

Any feature that has been described above in relation to any one aspect or embodiment of the invention is also disclosed hereby in relation to all other aspects and embodiments. Likewise, all combinations of two or more of the individual features or elements described above may be present in any aspect or embodiment. For brevity, all possible features and combinations have not been recited in relation to all aspects and embodiments, but they are expressly contemplated and hereby disclosed.

### EXAMPLES

### Ethical Approval

All protocols in the Examples were approved by the Committee for Ethics in Animal Experimentation of the University of Campinas (IACUC/CEEA-UNICAMP, Permit number 4754-1/2017).

### Materials

ACh, phenylephrine, guanethidine, atropine, were acquired from Sigma-Aldrich (St Louis, MO, USA). Human haptoglobin (Hp) solution was a kind gift from CSL Behring (Bern, Switzerland). Analytical grade was required for all reagents. Either deionized water was used as solvents, and working solutions were diluted prior to use.

### Animals and Treatment

All mice strains were originally purchased from Jackson Laboratories (Bar Harbor, ME). Characterization and breeding were performed at the Multidisciplinary Center for the Investigation of Biological Science in Laboratory Animals of University of Campinas. C57BL/6 male mice (wild-type, WT) and Berkeley transgenic SCD mice, aged 3 to 4 months-old, were used. Mice were housed three per cage on a 12 h light-dark cycle. SCD male mice were treated with treatment with haptoglobin (400 mg/kg, subcutaneous) or vehicle of Monday, Wednesday and Friday for a period of 1 month (24).

### Statistical Analysis

The GraphPad Prism Program (GraphPad Software Inc.) was used for statistical analysis. Data are expressed as the mean±SEM of N experiments. Statistical comparisons were made using Student's unpaired t-test. A value of P<0.05 was considered statistically significant.

### Functional Studies in Cavernosal Strips and Concentration-Response Curves

Strips of mouse corpus cavernosum were mounted in a 7-ml organ system containing Krebs solution at 37° C., continuously bubbled with a mixture of 95% O₂ and 5% CO₂ (pH 7.4), and suspended between two metal hooks. One hook was connected to a force transducer and the other acted as a fixed attachment point. Tissues were allowed to equilibrate for 60 min under a resting tension of 2.5 mN. Isometric force was recorded using a PowerLab 400™ data acquisition system (Software LabChart, version 7.0, AD Instrument, MA, USA). Cumulative concentration-response curves were constructed for the muscarinic agonist acetylcholine (ACh; 10⁻⁹ to 10⁻⁵ M) and sodium nitroprusside (SNP; 10⁻⁸ to 10⁻⁴ M) in cavernosal strips pre-contracted with phenylephrine (10⁻⁵ M). Cumulative concentration-response curves to the contractile agent phenylephrine (α1-adrenergic receptor agonist, 10⁻⁸ to 3×10⁻⁴ M) and KCl (3×10⁻⁴ to 3×10⁻¹ M) were obtained in cavernosal strips. Nonlinear regression analysis to determine the pEC₅₀ was carried out using GraphPad Prism (GraphPad Software, San Diego, CA, USA).

### Electrical-Field Stimulation (EFS)

EFS was applied on cavernosal strips placed between two platinum ring electrodes connected to a Grass S88 stimulator (Astro-Med Industrial Park, RI, USA). EFS was conducted at 50 V, 1 ms pulse width and trains of stimuli lasting 10 sec at varying frequencies. In order to study the nitrergic cavernosal relaxations, tissues were pretreated with guanethidine (3×10⁻⁵ M; to deplete the catecholamine stores of adrenergic fibers) and atropine (10⁻⁶ M; to produce muscarinic antagonism) for 30 minutes prior to pre-contraction with phenylephrine (10⁻⁵ M). When a stable contraction level was attained, a series of EFS-induced relaxations were constructed (2-32 Hz).

### Western Blot Analysis

Corpus cavernosum tissue was homogenized in lysis buffer and centrifuged at 12,000 g for 20 minutes at 4° C. Homogenates containing 50 g total proteins were run on 4-20% Tris-HCl gels (Bio-Rad Laboratories, Hercules, CA, USA) and transferred to a nitrocellulose membrane. Nonfat dry milk 5% (Bio-Rad) in Tris-buffered saline/Tween was used for 1 hour at 24° C. to block nonspecific binding sites. Membranes were incubated for 15-16 hour at 4° C. with the following antibodies: monoclonal anti-3-NT (1:1000, Abcam, Cambridge, MA), polyclonal anti-4-HNE antibody (1:1000, Abcam), polyclonal anti-p-eNOS (Ser-1177) antibody (1:1000, Abcam), polyclonal anti-eNOS antibody (1:1000, Abcam), polyclonal anti-nNOS antibody (1:1000, Abcam), polyclonal anti-PDE5 (1:500; Abcam), polyclonal anti-ROCK-1/ROCK-2 (1:1000; Abcam), gp91phox (1:1000; BD Transduction Laboratories, San Diego, CA) and monoclonal anti-β-actin (1:7000; Sigma-Aldrich, St. Louis, MO). Densitometry was analyzed using the Image J Software (National Institute of Health, Bethesda-MD, USA). Quantified densitometry results were normalized to β-actin. Quantified densitometry results of eNOS phosphorylated at Ser1177 were normalized to total eNOS.

Determination of cGMP Levels

Quantitative assays for cGMP were performed using a commercial enzyme immunoassay kit (Cayman Chemical Cyclic GMP EIA kit, Ann Arbor, MI, USA). For penile cGMP content, frozen penile tissue was homogenized in 5% trichloroacetic acid and centrifuged. TCA was extracted from the supernatant with three washes of water-saturated ether. cGMP was expressed as pmol/mg tissue.

### Example 1: Haptoglobin Treatment Corrects Exaggerated Corpus Cavernosum Relaxations from SCD Mouse

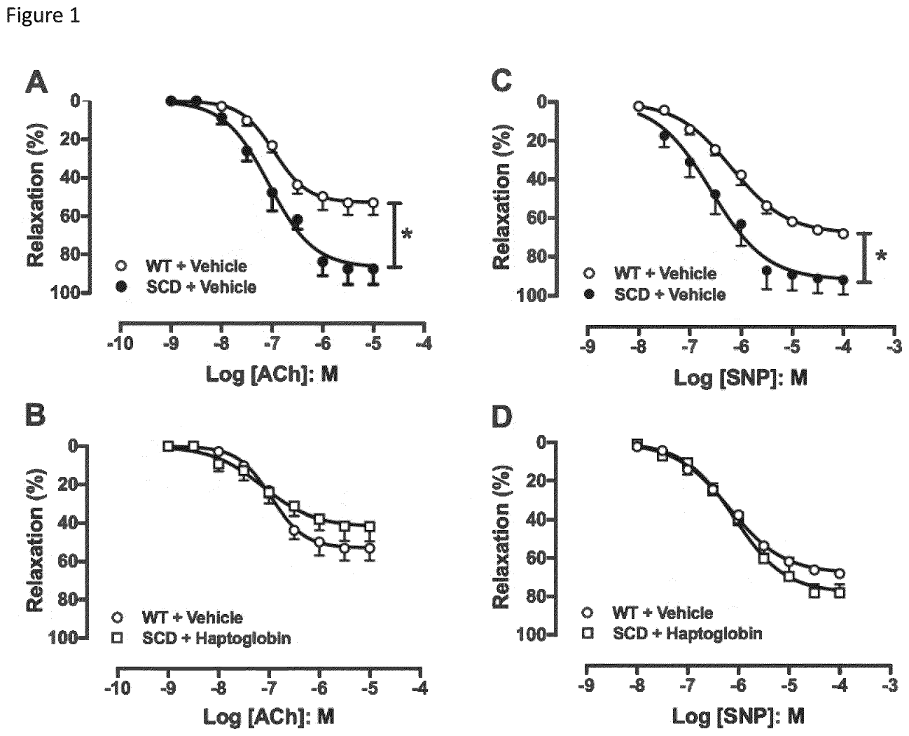

The cumulative addition of ACh (10⁻⁹ to 10⁻⁵ M) to PE-contracted tissues produced concentration-dependent relaxations in all groups (FIG. 1A, 1B). However, maximal response (Emax) produced by ACh were significantly higher in SCD compared with WT group (P<0.05; FIG. 1A; Table 1 below). Haptoglobin treatment of SCD mice reduced the Emax values of ACh (P<0.05; Table 1). No significant differences of potency (pEC50) for ACh were found among the groups. The pEC50 values for the ACh are shown in Table 1.

TABLE 1
Potency (pEC₅₀) and maximal responses (Eₘₐₓ) values obtained
from concentration-response curves in cavernosal strips from
WT and SCD mice treated with vehicle or haptoglobin.

| | | | | | SCD- | |
|---|---|---|---|---|---|---|
| | WT-Vehicle | | SCD-Vehicle | | Haptoglobin | |
| | | Eₘₐₓ | | Eₘₐₓ | | Eₘₐₓ |
| | pEC₅₀ | (%) | pEC₅₀ | (%) | pEC₅₀ | (%) |
| ACh | 6.96 ± | 53 ± 6 | 7.06 ± | 87 ± 7* | 7.12 ± | 42 ± 6# |
| | 0.07 | | 0.07 | | 0.14 | |
| SNP | 6.18 ± | 68 ± 1 | 6.57 ± | 92 ± 7* | 6.08 ± | 78 ± 4 |
| | 0.03 | | 0.10* | | 0.06# | |

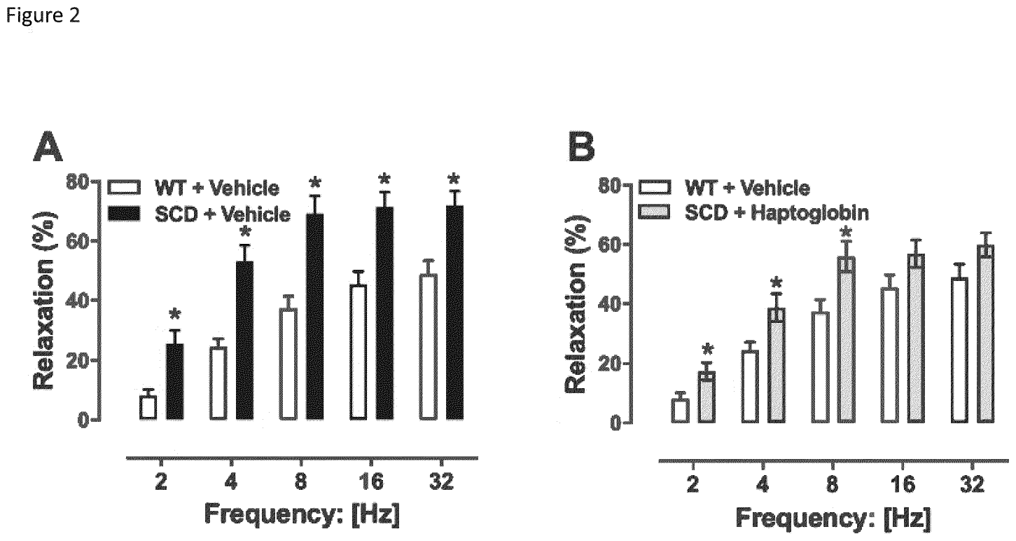

The cumulative addition of SNP (10⁻⁸ to 10⁻⁴ M) also produced concentration-dependent relaxations in all groups (FIG. 1C, 1D). The Emax produced by SNP was significantly higher (P<0.05) in corpus cavernosum from SCD compared to WT mice (FIG. 1C; Table 1), which was reduced by treatment with haptoglobin (FIG. 2D). SNP pEC50 value was significantly higher (P<0.05) in the corpus cavernosum of SCD compared to WT mice (FIG. 1C and Table 1), which was reversed (P<0.05) by treatment with haptoglobin (FIG. 1D and Table 1).

Electrical-field stimulation (EFS) of cavernosal tissues pretreated with guanethidine (3×10⁻⁵ M) and atropine (10⁻⁶ M) caused frequency-dependent mouse corpus cavernosum relaxations in all groups. Corpus cavernosum relaxations to EFS were significantly lower (P<0.05) in SCD compared to WT mice, as observed at 2 to 32 Hz (FIG. 2A). Haptoglobin treatment reduced the EFS-induced increased relaxant responses in corpus cavernosum from SCD (FIG. 2B).

The NO generated after erectile stimulation is the essential molecule that is produced in the penis to cause penile erection. NO is produced by the endothelium that lines the sinusoids of corpus cavernosum and also by nitrergic neurons (26). ACh promotes relaxation of corpus cavernosum by stimulating the production of NO by the endothelium. According to the results of Example 1, relaxation produced by ACh was greater in the corpus cavernosum of the SCD group. EFS promotes relaxation through neurogenic stimulation that results in NO production by nitrergic fibers (27). The relaxation produced by the EFS was also greater in the CC of the SCD group. The SNP is a NO-donor compound, which is used as a pharmacological tool to assess endothelium-independent relaxation (28). According to the results, SNP-induced relaxation was also higher in the SCD group. Therefore, when NO activates GCs, cGMP is produced, but it is not efficiently degraded by PDE5, thus cGMP accumulates in the smooth muscle cell promoting corpus cavernosum relaxation and excess penile erection in SCD (3). Long-term treatment with haptoglobin reduced relaxation induced by ACh, EFS and SNP in CC from SCD group. Without wishing to be bound by theory, the improvement in erectile function by haptoglobin treatment is likely due to normalization of PDE5 expression and decreased oxidative stress in the penises of SCD mice.

### Example 2: Haptoglobin Treatment Corrects Reduced Corpus Cavernosum Contractions from SCD Mouse

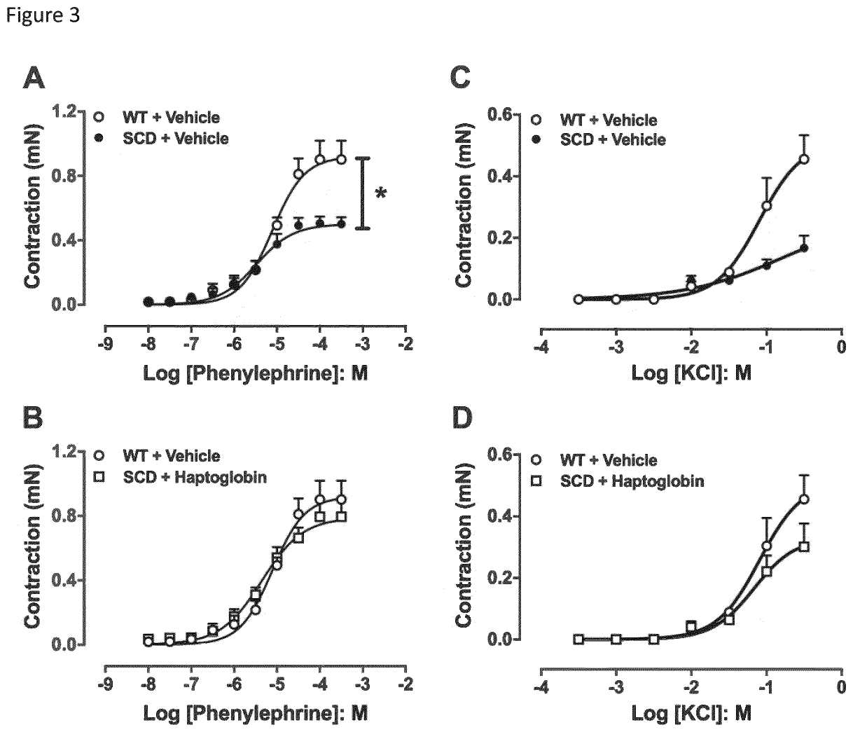

Phenylephrine (10⁻⁸ to 3×10⁻⁴ M) induced concentration-dependent corpus cavernosum contractions in all groups (FIG. 3A). The maximal responses (Emax) were significantly lower (P<0.05) in corpus cavernosum of SCD (0.50±0.08 mN) compared to WT mice (0.90±0.11 mN). Haptoglobin treatment of SCD mice increased the Emax values of phenylephrine in SCD group (0.79±0.11 mN).

In the evaluation of receptor-independent stimulation, cumulative addition of KCl produced concentration-dependent corpus cavernosum contractions in all groups (FIGS. 3C and 3D). The maximal responses (Emax) were significantly lower (P<0.05) in corpus cavernosum of SCD mice (0.16±0.04 mN) compared to WT mice (0.45±0.08 mN). Haptoglobin treatment of SCD mice increased the Emax values of KCl in SCD group (0.30±0.07 mN).

Noradrenaline released from the sympathetic nerve system induces contractions in corpus cavernosum due to postjunctional activation of α1-adrenoceptors coupled to Gq protein that activates phospholipase C, which catalyzes the cleavage of phosphatidylinositol into inositol trisphosphate and diacylglycerol, thus increasing intracellular calcium levels (34). Calcium produces smooth muscle contractions due to its binding to calmodulin which results in the activation of myosin light chain kinase (MLC), leading to phosphorylation of MLC (27). During this process, phosphorylated MLC interacts with beta-actin, resulting in smooth muscle contraction and maintenance of the penis in the flaccid state. (27,34). KCl promotes receptor-independent contraction, acts by depolarizing the smooth muscle cell membrane and promotes calcium influx (27). The inventors of the present invention have found that, surprisingly, contractions induced by the α1-adrenoceptor agonist and KCl were lower in SCD mice.

### Example 3: Haptoglobin Treatment Corrects Downregulated p-eNOS (Ser-1177) and eNOS, and does not Affect nNOS Protein Expression in the SCD Mouse Penis

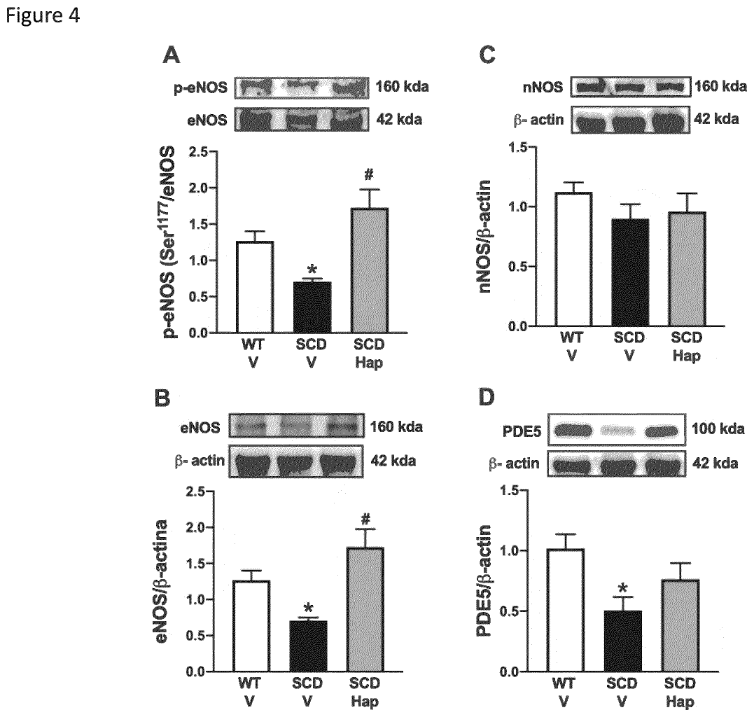

The protein expression for p-eNOS (Ser-1177) and eNOS and was significantly reduced (p<0.05) by approximately 45% and 44% in the penises of SCD-vehicle group compared to WT-vehicle group (FIGS. 4A and 4B, respectively). Haptoglobin treatment normalized (P<0.05) the protein level of p-eNOS (Ser-1177) and eNOS in penises from SCD group. Protein expression of nNOS was not modified by SCD or haptoglobin treatment (FIG. 4C).

According to the result of Example 3, the expression of total eNOS and phosphorylated eNOS at its positive regulatory site Ser-1177 was normalized by haptoglobin treatment in the SCD group, whereas no change was observed in the expression of nNOS. These results indicated that basal NO production was normalized in the penises of SCD mice.

### Example 4: Haptoglobin Treatment Increased PDE5 Protein Expressions in the SCD Mouse Penis

PDE5 protein expressions were significantly (p<0.05) decreased by approximately 50% in the penises of SCD-vehicle group compared to WT-vehicle group (FIG. 4D). Haptoglobin treatment increased the protein level of PDE5 by approximately 52% in the penises of SCD group (FIG. 4D).

Taken the results of Examples 3 and 4 together, it is demonstrated that improved endothelial function results in increased expression of PDE5. The increase in the expression of PDE5 in the smooth muscle of corpus cavernosum prevents the excessive relaxation induced by the stimulation of the NO-GCs pathway by ACh, EFS and SNP in SCD mice.

### Example 5: Haptoglobin Treatment Restores ROCK2, and does not Affect ROCK1 Protein Expressions in the SCD Mouse Penis

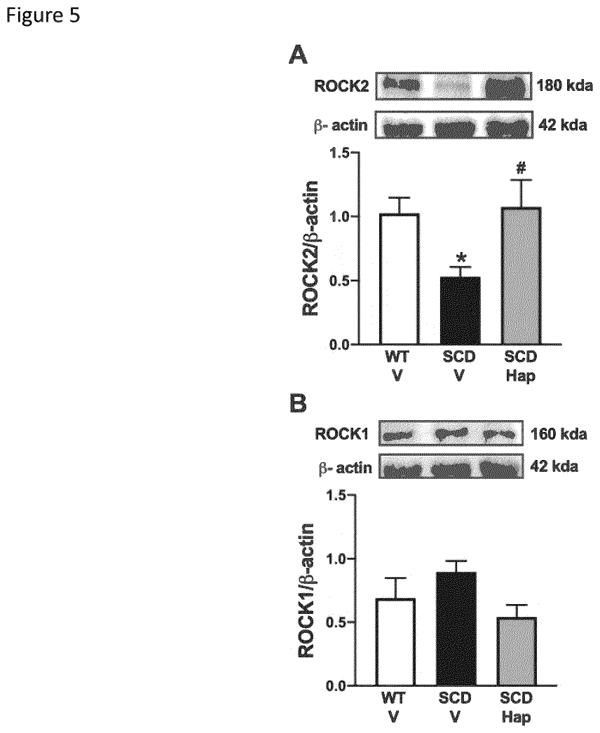

The protein expression for ROCK2 was significantly reduced (P<0.05) by approximately 48% in the penis of vehicle-treated SCD group in comparison with vehicle-treated WT mice (FIG. 5A). Treatment with haptoglobin fully restored the protein levels of ROCK2 in SCD group (FIG. 5A). Protein expression of ROCK1 was not modified by SCD or haptoglobin treatment compared to WT-vehicle group (FIG. 5B).

Activation of the RhoA/Rho-kinase signaling pathway acts by increasing Ca2+ sensitivity, thus participating in the contractile mechanism of corpus cavernosum smooth muscle. (35). There are two isoforms of ROCK, named ROCK1 and ROCK2 (35). The results of Example 5 showed that ROCK2 expression is lower in corpus cavernosum of SCD mice. The reduction of ROCK2 is in agreement with the results of Example 2, where contractions are reduced in SCD mice. Treatment with haptoglobin increased the contraction induced by phenylephrine and KCl, as well as the expression of ROCK2. Mice deficient for the eNOS enzyme display reduced NO bioavailability associated with lower RhoA/Rho-kinase activity in the penis, as well as a priapism phenotype (36). In previous examples, haptoglobin treatment restored eNOS expression in the SCD group, indicating that normalization of NO bioavailability in the endothelium is associated with increased ROCK2 expression in the penis.

### Example 6: Haptoglobin Treatment Corrects Increased Oxidative Stress and Upregulated Protein Expression of NADPH Oxidase Subunit Gp91Phox in the SCD Mouse Penis

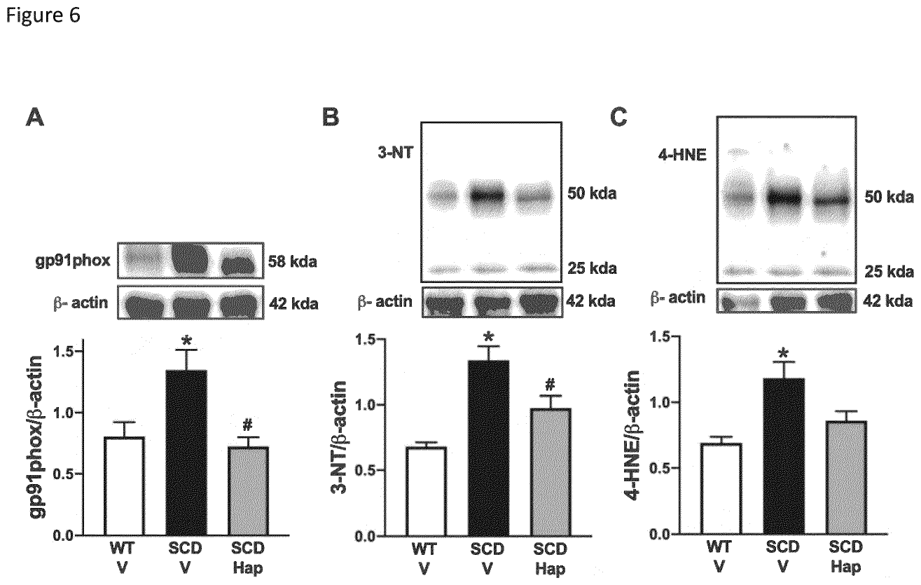

The protein expression of gp91phox was significantly higher (P<0.05) by approximately 67% in the penis of vehicle-treated SCD group in comparison with vehicle-treated WT mice (FIG. 6A). Treatment with haptoglobin fully restored the protein levels of gp91phox in SCD group (FIG. 6A).

The protein expression for 3-nitrotyrosine and 4-HNE was significantly higher (P<0.05) by approximately 95% and 75% in penile tissue from SCD in comparison with the WT group, respectively (FIGS. 6B and 6C). Haptoglobin treatment reduced the protein level of 3-nitrotyrosine and 4-HNE by approximately 27% and 28% in SCD group. (FIGS. 6B and 6C).

In this Example, haptoglobin treatment reduced gp91phox expression, indicating a lower production of superoxide anion in the penis. According to the result, the expression of markers of oxidative stress (4-HNE) and nitrosative stress (3-NT) was reduced by haptoglobin treatment. The expression of gp91phox is downregulated by a cGMP-dependent mechanism in the penis (42,43), whereas exogenous NO inhibits NADPH oxidase activity through direct s-nitrosylation of the p47phox subunit in human endothelial cells (44). In this example, the normalization of gp91phox subunit expression by haptoglobin treatment may involve improvement of endothelial function.

### Example 7: Haptoglobin Treatment Increased cGMP Levels in the SCD Mouse Penis

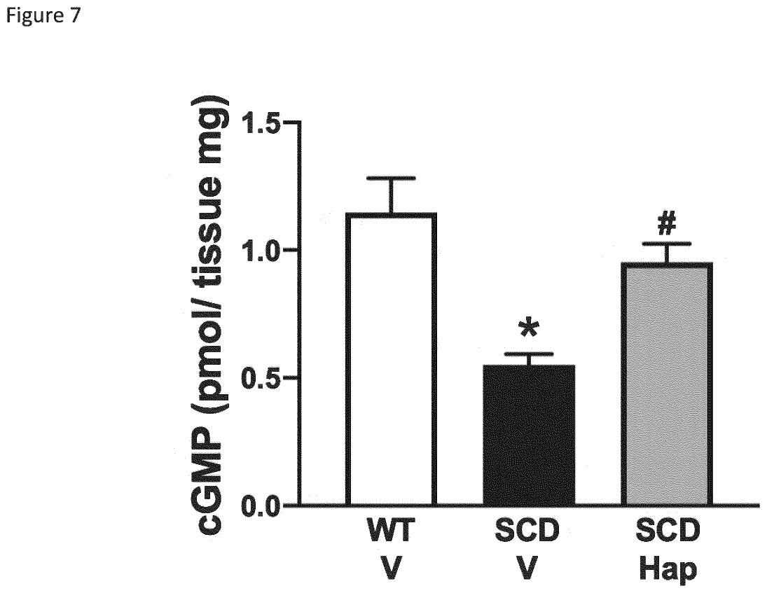

The basal cGMP content in the erectile tissue was 60% lower (P<0.05) in penises of SCD mice compared with WT-vehicle mice (FIG. 7). Haptoglobin treatment increased (P<0.05) the cGMP levels by approximately 72% in the penis of the SCD group (FIG. 7).

The above embodiments have been described by way of example only. Many other embodiments falling within the scope of the accompanying claims will be apparent to the skilled reader. Therefore, although the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

### REFERENCES

1. Telen M J, Malik P, Vercellotti G M. Therapeutic strategies for sickle cell disease: towards a multi-agent approach. Nat Rev Drug Discov. 2019 February; 18(2):139-58.
2. Salonia A, Eardley I, Giuliano F, Hatzichristou D, Moncada I, Vardi Y, et al. European Association of Urology guidelines on priapism. Eur Urol. 2014 February; 65(2):480-9.
3. Musicki B, Burnett A L. Mechanisms underlying priapism in sickle cell disease: targeting and key innovations on the preclinical landscape. Expert Opin Ther Targets. 2020 May; 24(5):439-50.
4. Adeyoju A B, Olujohungbe A B K, Morris J, Yardumian A, Bareford D, Akenova A, et al.

Priapism in sickle-cell disease; incidence, risk factors and complications—an international multicentre study. BJU Int. 2002 December; 90(9):898-902.
5. Mantadakis E, Cavender J D, Rogers Z R, Ewalt D H, Buchanan G R. Prevalence of priapism in children and adolescents with sickle cell anemia. J Pediatr Hematol Oncol. 1999 December; 21(6):518-22.
6. Alvaia M A, Maia H A A da S, Nelli A de M, Guimãries COS, Carvalho E S de S, Netto J M B, et al. Prevalence of priapism in individuals with sickle cell disease and implications on male sexual function. Einstein Sao Paulo Braz. 2020; 18:eAO5070.
7. Gladwin M T, Sachdev V. Cardiovascular abnormalities in sickle cell disease. J Am Coll Cardiol. 2012 Mar. 27; 59(13):1123-33.
8. Silva F H, Karakus S, Musicki B, Matsui H, Bivalacqua T J, Dos Santos J L, et al. Beneficial effect of the nitric oxide donor compound 3-(1,3-dioxoisoindolin-2-yl)benzyl nitrate on dysregulated phosphodiesterase 5, NADPH oxidase and nitrosative stress in the sickle cell mouse penis: implication for priapism treatment. J Pharmacol Exp Ther. 2016 Aug. 18;
9. Bivalacqua T J, Musicki B, Hsu L L, Berkowitz D E, Champion H C, Burnett A L. Sildenafil citrate-restored eNOS and PDE5 regulation in sickle cell mouse penis prevents priapism via control of oxidative/nitrosative stress. PloS One. 2013; 8(7):e68028.
10. Musicki B, Karakus S, Akakpo W, Silva F H, Liu J, Chen H, et al. Testosterone replacement in transgenic sickle cell mice controls priapic activity and upregulates PDE5 expression and eNOS activity in the penis. Andrology. 2018 January; 6(1):184-91.
11. Champion H C, Bivalacqua T J, Takimoto E, Kass D A, Burnett A L. Phosphodiesterase-5A dysregulation in penile erectile tissue is a mechanism of priapism. Proc Natl Acad Sci USA. 2005 Jan. 2; 102(5):1661-6.
12. Lagoda G, Sezen S F, Cabrini M R, Musicki B, Burnett A L. Molecular analysis of erection regulatory factors in sickle cell disease associated priapism in the human penis. J Urol. 2013 February; 189(2):762-8.
13. Kato G J. Priapism in sickle-cell disease: a hematologist's perspective. J Sex Med. 2012 January; 9(1):70-8.
14. Bivalacqua T J, Musicki B, Kutlu O, Burnett A L. New insights into the pathophysiology of sickle cell disease-associated priapism. J Sex Med. 2012 January; 9(1):79-87.
15. Bivalacqua T J, Ross A E, Strong T D, Gebska M A, Musicki B, Champion H C, et al.

Attenuated RhoA/Rho-kinase signaling in penis of transgenic sickle cell mice. Urology. 2010 August; 76(2):510.e7-12.
16. Reiter C D, Wang X, Tanus-Santos J E, Hogg N, Cannon R O, Schechter A N, et al. Cell-free hemoglobin limits nitric oxide bioavailability in sickle-cell disease. Nat Med. 2002 December; 8(12):1383-9.
17. Buehler P W, Humar R, Schaer D J. Haptoglobin Therapeutics and Compartmentalization of Cell-Free Hemoglobin Toxicity. Trends Mol Med. 2020 July; 26(7):683-97.
18. Muller-Eberhard U, Javid J, Liem H H, Hanstein A, Hanna M. Plasma concentrations of hemopexin, haptoglobin and heme in patients with various hemolytic diseases. Blood. 1968 November; 32(5):811-5.
19. Santiago R P, Guarda C C, Figueiredo C V B, Fiuza L M, Aleluia M M, Adanho C S A, et al.

Serum haptoglobin and hemopexin levels are depleted in pediatric sickle cell disease patients. Blood Cells Mol Dis. 2018 September; 72:34-6.
20. Cita K-C, Brureau L, Lemonne N, Billaud M, Connes P, Ferdinand S, et al. Men with Sickle Cell Anemia and Priapism Exhibit Increased Hemolytic Rate, Decreased Red Blood Cell Deformability and Increased Red Blood Cell Aggregate Strength. PloS One. 2016; 11(5):e0154866.
21. Kato G J, McGowan V, Machado R F, Little J A, Taylor J, Morris C R, et al. Lactate dehydrogenase as a biomarker of hemolysis-associated nitric oxide resistance, priapism, leg ulceration, pulmonary hypertension, and death in patients with sickle cell disease.

### Blood. 2006 Mar. 15; 107(6):2279-85.

22. Nolan V G, Wyszynski D F, Farrer L A, Steinberg M H. Hemolysis-associated priapism in sickle cell disease. Blood. 2005 Nov. 1; 106(9):3264-7.
23. Belcher J D, Chen C, Nguyen J, Abdulla F, Zhang P, Nguyen H, et al. Haptoglobin and hemopexin inhibit vaso-occlusion and inflammation in murine sickle cell disease: Role of heme oxygenase-1 induction. PloS One. 2018; 13(4):e0196455.
24. Shi P A, Choi E, Chintagari N R, Nguyen J, Guo X, Yazdanbakhsh K, et al. Sustained treatment of sickle cell mice with haptoglobin increases HO-1 and H-ferritin expression and decreases iron deposition in the kidney without improvement in kidney function. Br J Haematol. 2016 November; 175(4):714-23.
25. Schaer C A, Deuel J W, Schildknecht D, Mahmoudi L, Garcia-Rubio I, Owczarek C, et al.

Haptoglobin Preserves Vascular Nitric Oxide Signaling during Hemolysis. Am J Respir Crit Care Med. 2016 May 15; 193(10):1111-22.
26. Hurt K J, Sezen S F, Lagoda G F, Musicki B, Rameau G A, Snyder S H, et al. Cyclic AMP-dependent phosphorylation of neuronal nitric oxide synthase mediates penile erection.

### Proc Natl Acad Sci USA. 2012 Oct. 9; 109(41):16624-9.

27. Andersson K-E. Mechanisms of Penile Erection and Basis for Pharmacological Treatment of Erectile Dysfunction. Pharmacol Rev. 2011 Jan. 12; 63(4):811-59.
28. Kowaluk E A, Seth P, Fung H L. Metabolic activation of sodium nitroprusside to nitric oxide in vascular smooth muscle. J Pharmacol Exp Ther. 1992 September; 262(3):916-22.
29. Musicki B, Karakus S, La Favor J D, Chen H, Silva F H, Sturny M, et al. TSPO ligand FGIN-1-27 controls priapism in sickle cell mice via endogenous testosterone production. J Cell Physiol. 2020 Sep. 24;
30. Lin C-S, Chow S, Lau A, Tu R, Lue T F. Human PDE5A gene encodes three PDE5 isoforms from two alternate promoters. Int J Impot Res. 2002 February; 14(1):15-24.
31. Silva F H, Claudino M A, Calmasini F B, Alexandre E C, Franco-Penteado C, Burnett A L, et al. Sympathetic Hyperactivity, Increased Tyrosine Hydroxylase and Exaggerated Corpus Cavernosum Relaxations Associated with Oxidative Stress Plays a Major Role in the Penis Dysfunction in Townes Sickle Cell Mouse. PloS One. 2016; 11(12):e0166291.
32. Horst B G, Marletta M A. Physiological activation and deactivation of soluble guanylate cyclase. Nitric Oxide Biol Chem. 2018 Jul. 1; 77:65-74.
33. Hsu L L, Champion H C, Campbell-Lee S A, Bivalacqua T J, Manci E A, Diwan B A, et al. Hemolysis in sickle cell mice causes pulmonary hypertension due to global impairment in nitric oxide bioavailability. Blood. 2007 Apr. 1; 109(7):3088-98.
34. Mitidieri E, Cirino G, d'Emmanuele di Villa Bianca R, Sorrentino R. Pharmacology and perspectives in erectile dysfunction in man. Pharmacol Ther. 2020 Apr. 1; 208:107493.
35. Chitaley K, Wingard C J, Clinton Webb R, Branam H, Stopper V S, Lewis R W, et al. Antagonism of Rho-kinase stimulates rat penile erection via a nitric oxide-independent pathway. Nat Med. 2001 January; 7(1):119-22.
36. Bivalacqua T J, Liu T, Musicki B, Champion H C, Burnett A L. Endothelial nitric oxide synthase keeps erection regulatory function balance in the penis. Eur Urol. 2007 June; 51(6):1732-40.
37. Rifkind J M, Mohanty J G, Nagababu E. The pathophysiology of extracellular hemoglobin associated with enhanced oxidative reactions. Front Physiol. 2014; 5:500.
38. Schaer D J, Buehler P W, Alayash A I, Belcher J D, Vercellotti G M. Hemolysis and free hemoglobin revisited: exploring hemoglobin and hemin scavengers as a novel class of therapeutic proteins. Blood. 2013 Feb. 21; 121(8):1276-84.
39. Chintagari N Reddy, Nguyen J, Belcher J D, Vercellotti G M, Alayash A I. Haptoglobin attenuates hemoglobin-induced heme oxygenase-1 in renal proximal tubules cells and kidneys of a mouse model of sickle cell disease. Blood Cells Mol Dis. 2015 March; 54(3):302-6.
40. Musicki B, Liu T, Sezen S F, Burnett A L. Targeting NADPH oxidase decreases oxidative stress in the transgenic sickle cell mouse penis. J Sex Med. 2012 August; 9(8):1980-7.
41. Pacher P, Beckman J S, Liaudet L. Nitric oxide and peroxynitrite in health and disease. Physiol Rev. 2007 January; 87(1):315-424.
42. Nunes K P, Teixeira C E, Priviero F B M, Toque H A, Webb R C. Beneficial effect of the soluble guanylyl cyclase stimulator BAY 41-2272 on impaired penile erection in db/db−/− type II diabetic and obese mice. J Pharmacol Exp Ther. 2015 May; 353(2):330-9.
43. Teixeira C E, Priviero F B M, Webb R C. Effects of 5-Cyclopropyl-2-[1-(2-fluoro-benzyl)-1H-pyrazolo[3,4-b]pyridine-3-yl]pyrimidin-4-ylamine (BAY 41-2272) on Smooth Muscle Tone, Soluble Guanylyl Cyclase Activity, and NADPH Oxidase Activity/Expression in Corpus Cavernosum from Wild-Type, Neuronal, and Endothelial Nitric-Oxide Synthase Null Mice. J Pharmacol Exp Ther. 2007 Jan. 9; 322(3):1093-102.
44. Selemidis S, Dusting G J, Peshavariya H, Kemp-Harper B K, Drummond G R. Nitric oxide suppresses NADPH oxidase-dependent superoxide production by S-nitrosylation in human endothelial cells. Cardiovasc Res. 2007 Jul. 15; 75(2):349-58.
45. Lagoda G, Sezen S F, Hurt K J, Cabrini M R, Mohanty D K, Burnett A L. Sustained nitric oxide (NO)-releasing compound reverses dysregulated NO signal transduction in priapism. FASEB J. 2014 Jan. 1; 28(1):76-84.
46. Belcher J D, Chen C, Nguyen J, Milbauer L, Abdulla F, Alayash A I, et al. Heme triggers TLR4 signaling leading to endothelial cell activation and vaso-occlusion in murine sickle cell disease. Blood. 2014 Jan. 16; 123(3):377-90.
47. Kimmel J R. [70] Guanidination of proteins. Methods Enzymol. 1967; 11:584-589.
48. Haugen T H, Hanley J M, Heath E C. Haptoglobin. A novel mode of biosynthesis of a liver secretory glycoprotein. J Biol Chem. 1981; 256(3):1055-1057.
49. Misumi Y, Tanaka Y, Ikehara Y. Biosynthesis, intracellular processing and secretion of haptoglobin in cultured rat hepatocytes. Biochem Biophys Res Commun. 1983; 114(2):729-736.
50. Koch W, Latz W, Eichinger M, et al. Genotyping of the common haptoglobin Hp 1/2 polymorphism based on PCR. Clin Chem. 2002; 48(9):1377-1382.
51. Chapter 2—Haptoglobin Polymorphism and Infection; Advances in Clinical Chemistry, 2010, 50:23-46
52. Altschul S F, Gish W, Miller W, Myers E W, Lipman D J. Basic local alignment search tool. J Mol Biol. 1990; 215(3):403-410.
53. Morishita K, Ito N, Koda S, et al. Haptoglobin phenotype is a critical factor in the use of fucosylated haptoglobin for pancreatic cancer diagnosis. Clin Chim Acta. 2018; 487:84-89.
54. Cruickshank A M. ACP Best Practice No 166: CSF spectrophotometry in the diagnosis of subarachnoid haemorrhage. J Clin Pathol. 2001; 54(11):827-830.
55. Hugelshofer M, Sikorski C M, Seule M, et al. Cell-Free Oxyhemoglobin in Cerebrospinal Fluid After Aneurysmal Subarachnoid Hemorrhage: Biomarker and Potential Therapeutic Target. World Neurosurg. 2018; 120:e660-e666.
56. Schellenberger V, Wang C W, Geething N C, et al. A recombinant polypeptide extends the in vivo half-life of peptides and proteins in a tunable manner. Nat Biotechnol. 2009; 27:1186-1190.
57. Dumont J A, Low S C, Peters R T, Bitonti A J. Monomeric Fc Fusions. BioDrugs. 2006; 20:151-160.

1. A method of treating or preventing exaggerated erectile response and/or preventing permanent erectile dysfunction, comprising administering a haptoglobulin or a nucleic acid encoding haptoglobin to a subject.

2. The method of claim 1, wherein the exaggerated erectile response is priapism.

3. The method of claim 1, wherein the haptoglobin increases PDE5 protein expression.

4. The method of claim 1, wherein the haptoglobin decreases exaggerated corpus cavernosum relaxations.

5. The method of claim 1, wherein the haptoglobin is a human plasma haptoglobin.

6. The method of claim 1, wherein the haptoglobin or the nucleic acid encoding haptoglobin is administered parenterally.

7. The method of claim 1, wherein the haptoglobin or the nucleic acid encoding haptoglobin is administered to the subject in a dosage of from 0.5 g to 50 g per subject.

8. The method of claim 1, wherein the haptoglobin or the nucleic acid encoding haptoglobin is administered three times a week, for a period of one month or longer.

9. A method of treating or preventing exaggerated erectile response and/or preventing permanent erectile dysfunction, comprising administering a pharmaceutical composition to a subject, wherein the pharmaceutical composition comprises an adeno-associated viral (AAV) vector with a transgene encoding a haptoglobin gene.

10. The method of claim 9, wherein the adeno-associated viral (AAV) vector comprises an AAV2 serotype, an AAV5 serotype, an AAV9 serotype, or a combination thereof.

11. The method of claim 9, wherein the exaggerated erectile response is priapism.

12. The method of claim 1, wherein the subject has sickle cell disease (SCD).

13. The method of claim 2, wherein the priapism is associated with sickle cell disease (SCD).

14. The method of claim 6, wherein the haptoglobin or the nucleic acid encoding haptoglobin is administered parenterally by injection or infusion.

15. The method of claim 9, wherein the subject has sickle cell disease (SCD).

16. The method of claim 11, wherein the priapism is associated with sickle cell disease (SCD).


SEQUENCE LISTING (5 sequences)

SEQ ID NO: 1 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 2 - protein, 347 aa, Homo sapiens
MSALGAVIALLLWGQLFAVDSGNDVTDIADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 3 - protein, 245 aa, Homo sapiens
ILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 4 - protein, 142 aa, Homo sapiens
VDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQ

SEQ ID NO: 5 - protein, 83 aa, Homo sapiens
VDSGNDVTDIADDGCPKPPEIAHGYVEHSVRYQCKNYYKLRTEGDGVYTLNNEKQWINKAVGDKLPECEAVCGKPKNPANPVQ